(19) United States
(12) Patent Application Publication
(10) Pub. No.: US 2025/0288767 A1
(43) Pub. Date: Sep. 18, 2025

(54) STRUCTURE FOR WIRING CABLE TO ROTATING STRUCTURE, AND CPAP DEVICE

(71) Applicant: Murata Manufacturing Co., Ltd., Nagaokakyo-shi (JP)

(72) Inventor: Atsushi HATTORI, Nagaokakyo-shi (JP)

(21) Appl. No.: 19/223,110

(22) Filed: May 30, 2025

Related U.S. Application Data
(63) Continuation of application No. PCT/JP2023/042701, filed on Nov. 29, 2023.

(30) Foreign Application Priority Data
Nov. 30, 2022 (JP) 2022-191513

(51) Int. Cl. A61M 16/08; A61M 16/00; H01R 35/04

(52) U.S. Cl. CPC A61M 16/0816; A61M 16/0066; H01R 35/04

(57) ABSTRACT

A structure for wiring a cable to a rotating structure includes a non-rotating structure, a rotating structure which is arranged to be rotatable with respect to the non-rotating structure, and a cable which has one end connected to the non-rotating structure and the other end connected to the rotating structure. The non-rotating structure has a cavity portion allowing deformation of a routing shape of the cable during rotation of the rotating structure. The rotating structure has a main body around which the cable is wound, a cover which is arranged lateral to the main body in a manner to be separate from the main body, and a cavity portion which is formed between the main body and the cover and allows deformation of a routing shape of the cable during the rotation of the rotating structure.

### CROSS REFERENCE TO RELATED APPLICATION

This is a continuation of International Application No. PCT/JP2023/042701 filed on Nov. 29, 2023 which claims priority from Japanese Patent Application No. 2022-191513 filed on Nov. 30, 2022. The contents of these applications are incorporated herein by reference in their entireties.

### BACKGROUND OF THE DISCLOSURE

### Field of the Disclosure

The present disclosure relates to a structure which includes a non-rotating structure, a rotating structure, and a cable having one end connected to the non-rotating structure and the other end connected to the rotating structure.

### Description of the Related Art

Japanese Examined Patent Application Publication No. 62-5357 describes a device for transmitting driving force to a rotating body. The device of Japanese Examined Patent Application Publication No. 62-5357 includes a rotating cylinder, a rotary terminal, and a cable. The device of Japanese Examined Patent Application Publication No. 62-5357 winds the cable around the outer circumferential surface of the rotating cylinder in accordance with revolution of the rotating cylinder. In the device of Japanese Examined Patent Application Publication No. 62-5357, the cable is introduced into the rotating cylinder through the rotary terminal.

Japanese Unexamined Patent Application Publication No. 2-236977 describes a connector device. The device of Japanese Unexamined Patent Application Publication No. 2-236977 includes a cylindrical housing and a flat cable. The housing incorporates an annular cylindrical shaft portion having a gap in a portion in a circumferential direction. The device of Japanese Unexamined Patent Application Publication No. 2-236977 feeds and winds the flat cable through a gap of the cylindrical shaft portion.

### BRIEF SUMMARY OF THE DISCLOSURE

However, the conventional configurations as disclosed in Japanese Examined Patent Application Publication No. 62-5357 and Japanese Unexamined Patent Application Publication No. 2-236977 have points where a large load is applied to the cables during the feeding and winding the cables. Therefore, the cables may be disconnected.

Therefore, a possible benefit of the present disclosure is to suppress disconnection of a cable having one end connected to a non-rotating structure and the other end connected to a rotating structure.

A structure for wiring a cable to a rotating structure according to an embodiment of the present disclosure includes a non-rotating structure, a rotating structure which is arranged to be rotatable with respect to the non-rotating structure, and a cable which has one end connected to the non-rotating structure and the other end connected to the rotating structure.

The non-rotating structure has a first cavity portion allowing deformation of a routing shape of the cable during rotation of the rotating structure. The rotating structure has a main body having a side portion around which the cable is wound, a cover arranged separate from the side portion of the main body, and a second cavity portion formed between the main body and the cover and allowing deformation of the routing shape of the cable during the rotation of the rotating structure.

In this configuration, even when a distance between the one end of the cable and the other end of the cable changes when the rotating structure rotates with respect to the non-rotating structure, deflection of the cable caused by the change of the distance is accommodated by at least one of the first cavity portion and the second cavity portion. This suppresses application of an undesired stress to the cable.

According to the present disclosure, it is possible to suppress disconnection of the cable having one end connected to the non-rotating structure and the other end connected to the rotating structure.

### BRIEF DESCRIPTION OF THE SEVERAL VIEWS OF THE DRAWINGS

FIG. 1 is an external perspective view of a CPAP device according to an embodiment of the present disclosure.

FIG. 2 is an external perspective view of a base unit according to the embodiment of the present disclosure.

FIGS. 3A and 3B are plan views showing a portion of the base unit where a vent pipe is arranged.

FIG. 4A is a perspective view of the vent pipe as viewed from the front side, and FIG. 4B is an exploded perspective view of the vent pipe.

FIG. 5A is a perspective view of the vent pipe as viewed from the back side, and FIG. 5B is a partial side sectional view of the vent pipe.

FIG. 6A is an external perspective view of a main body of the vent pipe, and FIG. 6B is a plan view of the main body of the vent pipe.

FIG. 7A is an external perspective view of a front side of a cover of the vent pipe, and FIG. 7B is an external perspective view of a back side of the cover of the vent pipe.

FIG. 8A is a plan view showing a structure of arranging the vent pipe on a lid, and FIG. 8B is a drawing showing a state where the cover in FIG. 8A is removed.

FIGS. 9A and 9B are plan views each showing one aspect of the vent pipe during rotation.

FIG. 10 is a plan view showing one aspect of the vent pipe during rotation.

### DETAILED DESCRIPTION OF THE DISCLOSURE

A structure for wiring a cable to a rotating structure according to an embodiment of the present disclosure will be described with reference to the drawings. In the present embodiment, a CPAP device will be described as an example of an application target of the structure for wiring a cable to a rotating structure. However, the application target of the structure for wiring a cable to a rotating structure is not limited to the CPAP device.

### (Outline of CPAP Device 10)

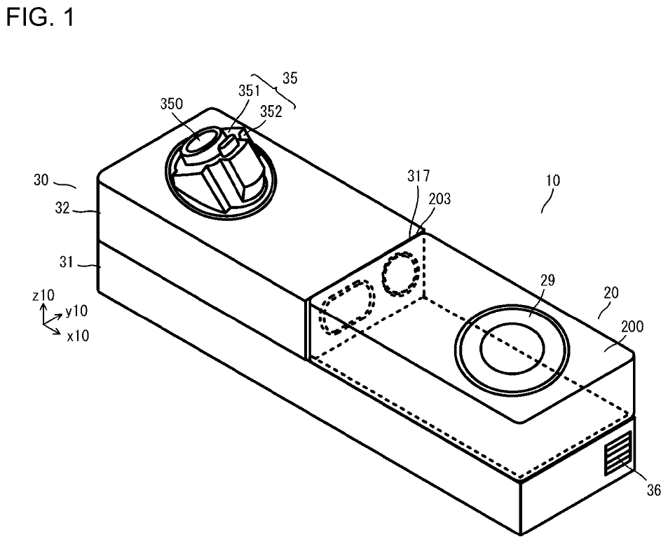

FIG. 1 is an external perspective view of the CPAP device according to the embodiment of the present disclosure. FIG. 1 shows a state in which a main unit is mounted on a base unit.

As shown in FIG. 1, a CPAP device 10 includes a main unit 20 and a base unit 30.

The outer shape of the main unit 20 is a rectangular parallelepiped, and the height is shorter than the length in the long direction and the length in the short direction. The rectangular parallelepiped in the present embodiment is not limited to a perfect rectangular parallelepiped, but includes a rectangular parallelepiped having various chamfered apexes and edges.

The base unit 30 has a rectangular shape having a long direction and a short direction in plan view. The base unit 30 has a taller portion and a shorter portion, and the taller portion is higher than the shorter portion. That is, the outer shape of the base unit 30 is a shape obtained by connecting a rectangular parallelepiped taller portion and a rectangular parallelepiped shorter portion to each other in the long direction of the base unit 30.

The main unit 20 can be mounted on the upper surface of the shorter portion of the base unit 30. The outer shape of the CPAP device 10 is a rectangular parallelepiped in a state where the main unit 20 is mounted on the base unit 30.

In the state where the main unit 20 is mounted on the base unit 30 (mounted state), the flow path of the gas of the main unit 20 and the flow path of the gas of the base unit 30 communicate with each other. A first electric circuit of the main unit 20 and a second electric circuit of the base unit 30 are electrically connected to each other. Thus, the main unit 20 and the base unit 30 cooperate to realize the function of the CPAP device 10. In a state where the main unit 20 is detached from the base unit 30, the main unit 20 operates independently to realize the function of the CPAP device 10.

The main unit 20 incorporates a blower, which is not shown. The CPAP device 10 generates an air flow to be supplied to a user by driving the blower.

### (Base Unit 30)

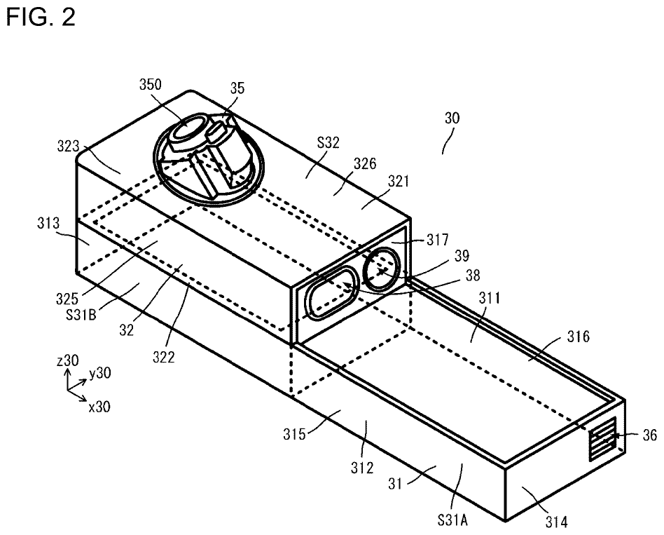

FIG. 2 is an external perspective view of the base unit according to the embodiment of the present disclosure. The base unit 30 corresponds to a main body of the CPAP device 10 in the present disclosure.

As shown in FIG. 2, the base unit 30 includes a box body 31 and a lid 32. Schematically, the box body 31 is partially open on the upper surface side. The lid 32 is rotatably connected to the box body 31.

### (Box Body 31)

The box body 31 includes an upper wall 311, a lower wall 312, a side wall 313, a side wall 314, a side wall 315, a side wall 316, and a side wall 317. The box body 31 has a space S31A formed by the upper wall 311, the lower wall 312, the side wall 314, the side wall 315, the side wall 316, and the side wall 317. Further, the box body 31 has a space S31B formed by the upper wall 311, the lower wall 312, the side wall 313, the side wall 315, the side wall 316, and the side wall 317.

The space S31B has an opening between the side wall 317 and the side wall 313 in plan view on the upper surface side where the upper wall 311 is arranged. The portion forming this space S31B accommodates a humidification chamber (not shown).

The side wall 314 has an intake port 36. The intake port 36 penetrates the side wall 314 in the thickness direction. The side wall 317 has an exhaust port 38 and an intake port 39 above a portion connected with the upper wall 311. The exhaust port 38 penetrates the inside of the side wall 317 and is coupled to the space S31A. The intake port 39 penetrates the side wall 317 in the thickness direction.

### (Lid 32)

The lid 32 includes an upper wall 321, a side wall 323, a side wall 325, and a side wall 326. The lid 32 has a space S32 formed by the upper wall 321, the side wall 323, the side wall 325, and the side wall 326. The lid 32 corresponds to a non-rotating structure of the present disclosure.

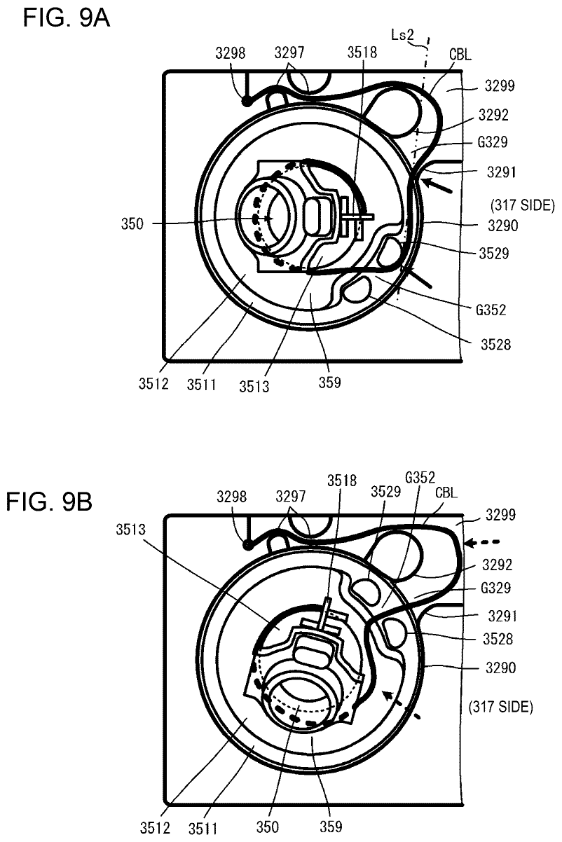

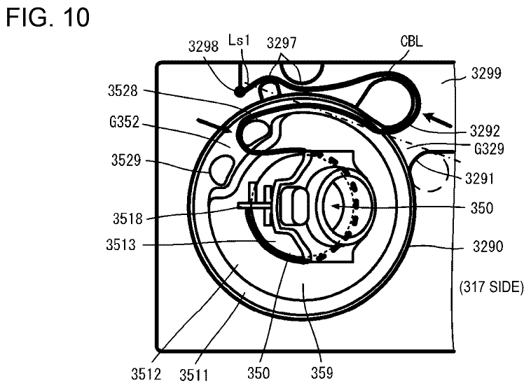

A vent pipe 35 is arranged on the upper wall 321. The vent pipe 35 has a shape protruding outward from the upper wall 321. The vent pipe 35 has a vent hole 350. The vent pipe 35 is arranged to be rotatable by 180° in plan view (see FIGS. 9A, 9B, and 10). The vent pipe 35 corresponds to the rotating structure of the present disclosure. The specific configuration of the vent pipe 35 will be described later.

With such a structure, the CPAP device 10 generates an air flow by the blower of the main unit 20, intakes gas from the intake port 36 of the base unit 30, and delivers the gas from the vent pipe 35. At this time, the CPAP device 10 controls the driving of the blower and heats water in a storage tank with a heater, thereby delivering an air flow having a desired flow rate, delivery pressure, and humidity.

The vent pipe 35 is physically and electrically connected to an inhalation mask worn by a user through a pipe (not shown). The air flow delivered from the CPAP device 10 is thereby provided to the user.

### (Structure for Wiring Cable to Vent Pipe (Structure for Wiring Cable to Rotating Structure))

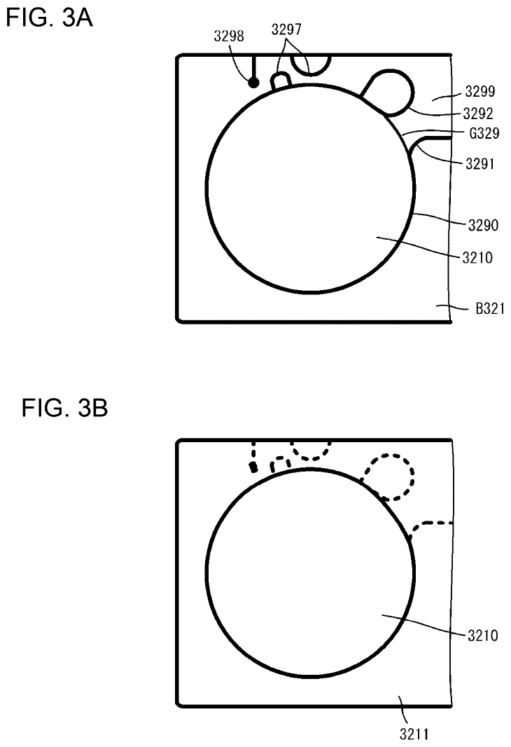

FIGS. 3A and 3B are plan views showing a portion of the base unit where the vent pipe is arranged. FIG. 3A shows a state where the upper cover is removed, and FIG. 3B shows a state where an upper cover is attached.

### (Lid of Base Unit (Non-Rotating Structure))

As shown in FIGS. 3A and 3B, the lid 32 of the base unit 30 includes a main body B321 and a main body cover 3211. The lid 32 corresponds to a non-rotating structure of the present disclosure.

The main body B321 has a concave portion whose upper surface side is open. As shown in FIGS. 3A and 3B, the main body B321 has an opening 3210 that is circular in plan view. The main body cover 3211 is plate-shaped, and has an opening which has substantially the same shape as the outer shape of a base 3522 (described in detail later) of the vent pipe 35. In plan view, the opening of the main body cover 3211 overlaps (communicates with) the opening 3210 of the main body B321, and is a smaller opening than the opening 3210. The main body cover 3211 covers the opening on the upper surface side of the main body B321 in this state. The lid 32 accordingly has a space of a predetermined height outside the opening 3210. A hole communicating with the vent hole 350 of the vent pipe 35 may be opened in the center of the concave portion of the opening 3210.

The main body B321 has an annular peripheral wall 3290 surrounding the opening 3210. The peripheral wall 3290 is cut in the middle in the circumferential direction. The cut portion of the peripheral wall 3290 is a guide opening G329.

A guide wall 3291 is formed at one end of the cut portion of the peripheral wall 3290. The guide wall 3291 has an arc shape in plan view. The guide wall 3291 has, for example, an arc shape of approximately ¼ of the circumference, and has an arc shape which bulges toward the guide opening G329 side.

One end of the guide wall 3291 is connected to one end of the cut portion of the peripheral wall 3290. The connecting portion has an arc shape. In other words, one end of the guide wall 3291 is connected to the peripheral wall 3290 without having an angular shape.

The other end of the guide wall 3291 is connected to a wall extending in a direction opposite to the peripheral wall 3290 side with respect to the guide wall 3291. The guide wall 3291 corresponds to a first guide wall of the present disclosure.

A guide wall 3292 is formed at the other end of the cut portion of the peripheral wall 3290. The guide wall 3292 has a substantially arc shape in plan view.

One end of the guide wall 3292 is connected to the other end of the cut portion of the peripheral wall 3290. The connecting portion has an arc shape. In other words, one end of the guide wall 3292 is connected to the peripheral wall 3290 without having an angular shape. The other end of the guide wall 3292 is connected to the outer surface of the peripheral wall 3290. The guide wall 3292 corresponds to a second guide wall of the present disclosure.

The guide wall 3291 and the guide wall 3292 have the above-described structure and are connected to the peripheral wall 3290 as described above, which forms a cavity portion 3299 having a large area in plan view outward from the guide opening G329 between the guide wall 3291 and the guide wall 3292. The cavity portion 3299 corresponds to a first cavity portion of the present disclosure.

The size of the cavity portion 3299 is, functionally speaking, a size capable of accommodating slack of a cable CBL described later, in other words, a size which allows deformation of a routing shape of the cable CBL outside the guide opening G329 (see FIG. 9B, for example).

The main body B321 further includes a plurality of protruding walls 3297 and a cable connection portion 3298. The plurality of protruding walls 3297 and the cable connection portion 3298 are arranged at positions on the outer side in the main body B321 and on the side close to the guide wall 3292 in the direction along the peripheral wall 3290. In the circumferential direction, the plurality of protruding walls 3297 and the cable connection portion 3298 are arranged in this order from the guide wall 3292 side.

### (Vent Pipe (Rotating Structure))

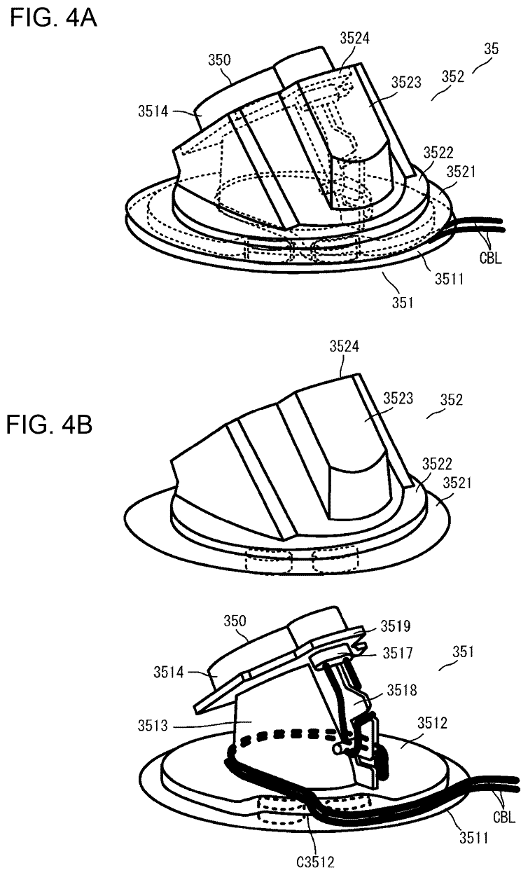

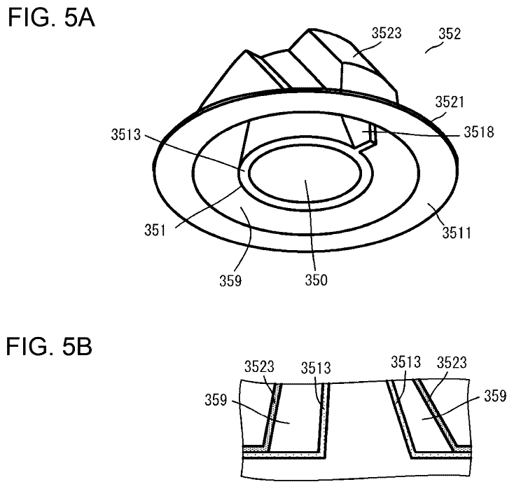

FIG. 4A is a perspective view of the vent pipe as viewed from the front side, and FIG. 4B is an exploded perspective view of the vent pipe. FIG. 5A is a perspective view of the vent pipe as viewed from the back side, and FIG. 5B is a partial side sectional view of the vent pipe.

### (Schematic Structure of Vent Pipe)

As shown in FIGS. 4A, 4B, 5A, and 5B, the vent pipe 35 includes a main body 351 and a cover 352. The vent pipe 35 corresponds to the rotating structure of the present disclosure. Schematically, the cover 352 covers the side of the main body 351. The cover 352 and the main body 351 are separated from each other, and a cavity portion 359 is formed between the cover 352 and the main body 351. The cavity portion 359 corresponds to a second cavity portion of the present disclosure.

The size of the cavity portion 359 is, functionally speaking, a size capable of accommodating slack of the cable CBL described later, in other words, a size which allows deformation of the routing shape of the cable CBL inside the vent pipe 35 (see FIG. 9B, for example).

### (Main Body of Vent Pipe)

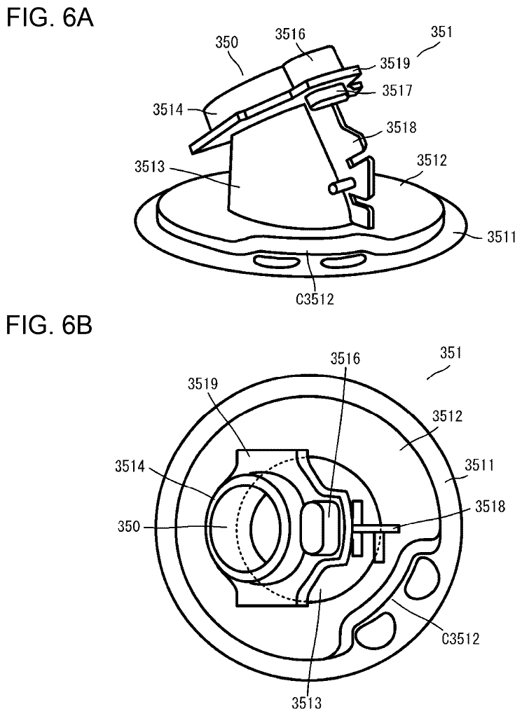

FIG. 6A is an external perspective view of the main body of the vent pipe, and FIG. 6B is a plan view of the main body of the vent pipe.

As shown in FIGS. 6A and 6B, the main body 351 includes a substrate 3511, a base 3512, a main wall 3513, an external connection wall 3514, an external terminal housing 3516, an internal connection terminal 3517, a cable fixing portion 3518, and a vent pipe lid 3519.

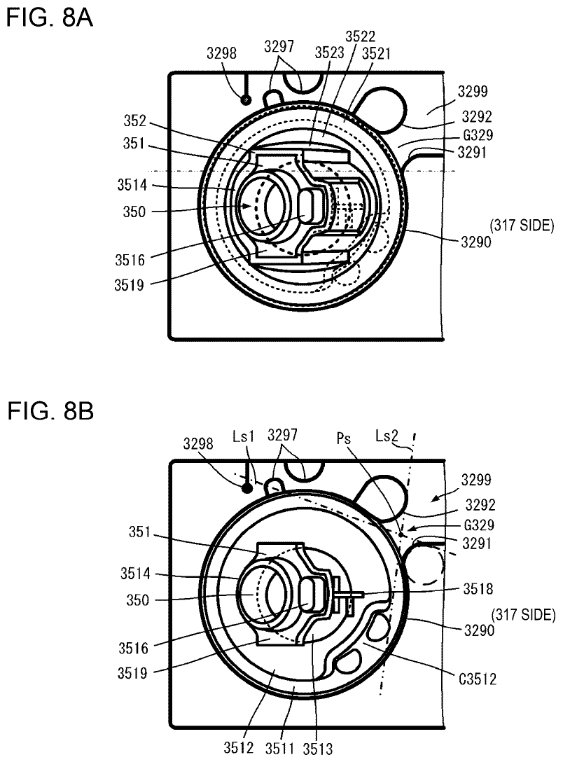

The substrate 3511 is an annular flat pate. The shape (outer shape) of the outer circumferential end of the substrate 3511 is substantially the same as the shape of the opening 3210 (see FIGS. 3A, 3B, 8A, and 8B, for example) of the main body B321 of the lid 32. Here, the substrate 3511 has a size that can be accommodated in the opening 3210.

The base 3512 is substantially circular in plan view. The outer shape of the base 3512 is smaller than the outer shape of the substrate 3511. The base 3512 is arranged above the substrate 3511. The outer circumferential end of the base 3512 is connected to the inner circumferential end of the substrate 3511. A concave portion C3512 which is concave inward in plan view is formed on the outer circumference of the base 3512.

The main wall 3513 has a cylindrical shape having an internal space. One end in the extending direction of the cylindrical shape of the main wall 3513 is connected to the upper surface of the base 3512. The extending direction of the main wall 3513 is not orthogonal to the upper surface of the base 3512, but has a predetermined angle with respect to the upper surface. The internal space of the main wall 3513 communicates with the opening of the inner portion of the base 3512 to which the main wall 3513 is connected. The wall forming the cylindrical shape of the main wall 3513 corresponds to a side portion of the rotating structure of the present disclosure.

The external connection wall 3514 has a cylindrical shape having an internal space. The extending direction of the external connection wall 3514 is substantially the same as the extending direction of the main wall 3513. One end in the extending direction of the cylindrical shape of the external connection wall 3514 is connected to the other end in the extending direction of the cylindrical shape of the main wall 3513.

The internal space of the external connection wall 3514 communicates with the internal space of the main wall 3513. These internal spaces form the vent hole 350 of the vent pipe 35.

The vent pipe lid 3519 is arranged at a connection portion between the main wall 3513 and the external connection wall 3514. The vent pipe lid 3519 is a flat plate. The vent pipe lid 3519 has a shape expanding in a radial direction with respect to the central axis of the cylindrical shape of the main wall 3513 and the external connection wall 3514.

The external terminal housing 3516 is formed to be connected to the peripheral surface of the external connection wall 3514. The external terminal housing 3516 includes an electrical external connection terminal.

The internal connection terminal 3517 is arranged on the opposite side from the external terminal housing 3516 with the vent pipe lid 3519 interposed therebetween. The internal connection terminal 3517 includes an electrical internal connection terminal. The internal connection terminal 3517 is electrically connected to the electrical external connection terminal of the external terminal housing 3516.

The cable fixing portion 3518 is a flat plate. The cable fixing portion 3518 has a shape protruding outward from the peripheral surface of the main wall 3513, and has a plate shape parallel to the extending direction of the cylindrical shape of the main wall 3513. The cable fixing portion 3518 is arranged at substantially the same position as the internal connection terminal 3517 in the circumferential direction of the main wall 3513. The cable fixing portion 3518 is formed with at least one groove or rod. The cable fixing portion 3518 may be formed on the cover 352.

### (Cover of Vent Pipe)

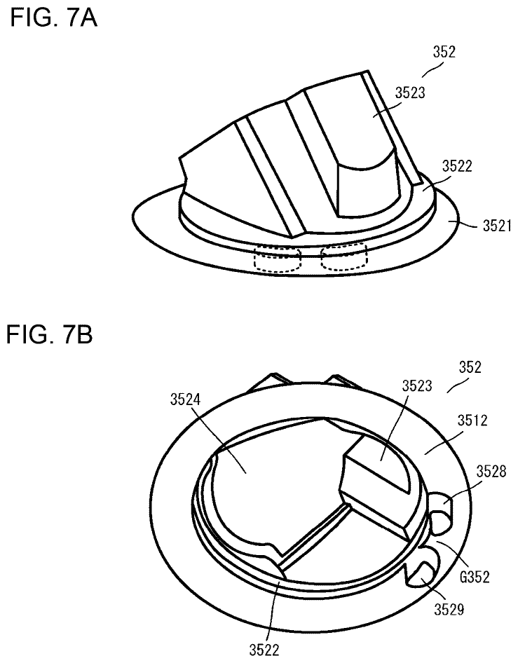

FIG. 7A is an external perspective view of the front side of the cover of the vent pipe, and FIG. 7B is an external perspective view of the back side of the cover of the vent pipe.

The cover 352 includes a substrate 3521, the base 3522, a cylindrical body 3523, a protruding portion 3528, and a protruding portion 3529.

The substrate 3521 is an annular flat plate. The shape (outer shape) of the outer circumferential end of the substrate 3521 is substantially the same as the shape of the opening 3210 (see FIGS. 3A, 3B, 8A, and 8B, for example) of the main body B321 of the lid 32 and the shape of the substrate 3511 of the main body 351.

The base 3522 is substantially circular in plan view. The outer shape of the base 3522 is smaller than the outer shape of the substrate 3521. The base 3522 is arranged above the substrate 3521. The outer circumferential end of the base 3522 is connected to the inner circumferential end of the substrate 3521.

The cylindrical body 3523 has an internal space 3524. One end in the extending direction of the cylindrical body 3523 is connected to the base 3522. The opening cross-sectional area of the other end in the extending direction of the cylindrical body 3523 is the same as the planar area of the vent pipe lid 3519 of the main body 351, and the opening shape is the same as the outer shape of the vent pipe lid 3519. The cross-sectional area of the internal space 3524 is substantially the same regardless of the position in the extending direction of the cylindrical body 3523.

The protruding portion 3528 and the protruding portion 3529 protrude from the rear surface of the substrate 3521. The shapes of the protruding portion 3528 and the protruding portion 3529 in plan view are flat ellipse. The shapes of the protruding portion 3528 and the protruding portion 3529 are not limited to the above-mentioned shapes, and any shapes having no corners on peripheral surfaces may be employed.

The protruding portion 3528 and the protruding portion 3529 are arranged at an interval along the circumferential direction of the substrate 3521. A space between the protruding portion 3528 and the protruding portion 3529 is a guide opening G352 on the vent pipe 35 side.

### (Arrangement of Main Body and Cover)

The cover 352 is arranged on the main body 351 such that the main wall 3513 of the main body 351 is accommodated in the internal space 3524 of the cylindrical body 3523 of the cover 352. The substrate 3521 of the cover 352 faces the substrate 3511 of the main body 351 with a distance therebetween, and the base 3522 of the cover 352 faces the base 3512 of the main body 351.

A space formed by the substrate 3521 of the cover 352 and the substrate 3511 of the main body 351 facing each other and a space formed by the base 3522 of the cover 352 and the base 3512 of the main body 351 facing each other communicate with each other through the concave portion C3512 of the base 3512 of the main body 351.

The protruding portion 3528 and the protruding portion 3529 of the cover 352 are arranged in the concave portion C3512.

The opening at the other end of the cylindrical body 3523 of the cover 352 is closed by the vent pipe lid 3519 of the main body 351.

With such a configuration, the vent pipe 35 has the cavity portion 359 between the main wall 3513 of the main body 351 and the cylindrical body 3523 of the cover 352 (see FIGS. 4A, 4B, 5A, 5B, and 8B).

The cavity portion 359 communicates with the outside from the outer circumferential end of the vent pipe 35 through a space between the base 3512 and the base 3522, a space of the concave portion C3512, and a space between the substrate 3511 and the substrate 3521.

### (Aspect of Cable Wiring in Vent Pipe)

As shown in FIGS. 4A and 4B, in the vent pipe 35, the end portion of the cable CBL on the vent pipe side is connected to the internal connection terminal 3517 and is routed along the extending direction of the main wall 3513. The cable CBL is routed in the circumferential direction of the main wall 3513 while being fixed by the groove and the protrusion of the cable fixing portion 3518. The cable CBL is guided substantially along the circumferential direction of the main wall 3513 to the guide opening G352 between the protruding portion 3528 and the protruding portion 3529. The cable CBL is routed from the guide opening G352 to the space between the substrate 3511 and the substrate 3521 and is drawn out to the outside of the vent pipe 35.

### (Aspect of Vent Pipe Arrangement on Lid and Rotation)

FIG. 8A is a plan view showing a structure of arranging the vent pipe on the lid, and FIG. 8B is a drawing showing a state where the cover in FIG. 8A is removed. Each of FIGS. 9A, 9B, and 10 is a plan view showing one aspect of the vent pipe during rotation.

The vent pipe 35 is accommodated in the opening 3210 of the lid 32. The vent pipe 35 is arranged to be rotatable with respect to the lid 32.

More specifically, in plan view of the lid 32, the vent pipe 35 is rotatably arranged on the lid 32 such that the external opening of the vent hole 350 faces the side wall 325 side, with the aspect in which the external opening of the vent hole 350 faces the side wall 323 side (the aspect in FIG. 9A) as one end of rotation and the aspect in which the external opening of the vent hole 350 faces the side opposite to the side wall 323 (the side wall 317 side) (the aspect in FIG. 10) as the other end of the rotation.

The cable CBL drawn from the vent pipe 35 is guided into the lid 32 through the guide opening G329 of the lid 32. The cable CBL is routed between the plurality of protruding walls 3297 to be connected and fixed to the cable connection portion 3298.

The lid 32 has the cavity portion 3299 and the vent pipe 35 has the cavity portion 359, whereby the routing shape of the cable CBL is deformed in accordance with the rotation of the vent pipe 35 as described below.

In the aspect in FIG. 9A, the cable CBL is wound around the peripheral surface of the main wall 3513 of the main body 351 of the vent pipe 35, passes through the cavity portion 359 of the vent pipe 35 and the space in the concave portion C3512, is abutted and bent on the peripheral surface of the protruding portion 3529, and is guided to the space between the substrate 3511 and the substrate 3521. Then, the cable CBL is bent along the peripheral surface of the guide wall 3291 and is guided into the cavity portion 3299 through the guide opening G329.

The peripheral surface of the protruding portion 3529 and the peripheral surface of the guide wall 3291 are smooth (without corners), whereby the cable CBL is not sharply bent. The disconnection of the cable CBL is accordingly suppressed.

In the aspect in FIG. 9B, the winding state of the cable CBL around the peripheral surface of the main wall 3513 is loosened by the rotation of the vent pipe 35. In this case, slack is generated in the cable CBL. This slack is accommodated to some extent in the cavity portion 359. Thus, buckling of the cable CBL is suppressed.

Further, when the slack of the cable CBL cannot be allowed only by the cavity portion 359, the cable CBL is pushed out to the outside of the vent pipe 35. The lid 32 has the cavity portion 3299 and the slack of the cable CBL pushed out from the vent pipe 35 is accordingly accommodated in the cavity portion 3299. Thus, the buckling of the cable CBL is more reliably suppressed.

In the aspect in FIG. 10, the cable CBL is wound again around the peripheral surface of the main wall 3513 of the main body 351 of the vent pipe 35, passes through the cavity portion 359 of the vent pipe 35 and the space in the concave portion C3512, is abutted and bent on the peripheral surface of the protruding portion 3528, and is guided to the space between the substrate 3511 and the substrate 3521. Then, the cable CBL is bent along the peripheral surface of the guide wall 3292 and is guided into the cavity portion 3299 through the guide opening G329.

The peripheral surface of the protruding portion 3528 and the peripheral surface of the guide wall 3292 are smooth (without corners), whereby the cable CBL is not sharply bent. The disconnection of the cable CBL is accordingly suppressed.

Thus, the vent pipe 35 and the lid 32 have the above-described configuration. Therefore, even when the vent pipe 35 rotates with respect to the lid 32, buckling or the like of the cable CBL is suppressed and disconnection of the cable CBL is suppressed.

In the above-described configuration, the end portion of the cable CBL on the vent pipe 35 side is connected to the internal connection terminal 3517 in a state of being bent in the extending direction of the main wall 3513, unlike the peripheral surface of the main wall 3513 around which the cable CBL is wound. In addition, the cable CBL is fixed by the cable fixing portion 3518 at the bent position on the main wall 3513. This alleviates the stress of the cable CBL at the connection portion on the vent pipe 35 side caused by the rotation of the vent pipe 35. As a result, disconnection of the cable CBL at the connection portion on the vent pipe 35 side is suppressed.

Further, in the above-described configuration, the end portion of the cable CBL on the lid 32 side is fixed through the plurality of protruding walls 3297. The outer surfaces of the plurality of protruding walls 3297 are arc-shaped. This alleviates the stress of the cable CBL at the connection portion on the lid 32 side caused by the rotation of the vent pipe 35. As a result, disconnection of the cable CBL at the connection portion on the lid 32 side is suppressed.

The vent pipe 35 and the lid 32 have the following relationship.

The guide wall 3292 does not overlap with a first common circumscribed line Ls1 (the dash-dotted line in FIG. 8B) between a virtual circle (the broken line (first virtual circle) in FIG. 8B) including an arc shape of the peripheral surface of the guide wall 3291 and a circle (second virtual circle) including a side surface (peripheral surface connecting the outer circumferential end of the base 3512 of the main body 351 and the inner circumferential end of the substrate 3511), with which the cable CBL is brought into contact, of the vent pipe 35. This suppresses undesired contact of the cable CBL to the guide wall 3292 when the vent pipe 35 rotates.

The guide wall 3291 does not overlap with a second common circumscribed line Ls2 (two dot chain line in FIG. 8B) between an arc (third virtual circle) of the peripheral surface of the guide wall 3292 and the circle (second virtual circle) including the side surface (peripheral surface connecting the outer circumferential end of the base 3512 of the main body 351 and the inner circumferential end of the substrate 3511), with which the cable CBL is brought into contact, of the vent pipe 35. This suppresses undesired contact of the cable CBL to the guide wall 3291 when the vent pipe 35 rotates.

Thus, application of an undesired stress to the cable CBL during the rotation of the vent pipe 35 is suppressed. As a result, buckling and disconnection of the cable CBL are suppressed.

For example, as shown in FIG. 8B, the cavity portion 359 and the cavity portion 3299 are formed in regions of vertical angles as an intersection point Ps of the first common circumscribed line Ls1 and the second common circumscribed line Ls2. This suppresses undesired collision of the cable CBL with the wall of the lid 32 and the wall of the vent pipe 35 when the cable CBL is routed between the cavity portion 359 and the cavity portion 3299. Therefore, application of an undesired stress to the cable CBL is suppressed.

In addition, it is preferable that the height or width of a space (corresponding to a tubular portion of the present disclosure) formed by the side surface (peripheral surface) of the base 3512, the substrate 3511, the substrate 3521, and the peripheral wall 3290 is three times or less the width of the cable CBL. This suppresses undesired bending of the cable CBL in the space formed between the substrate 3511 and the substrate 3521. Here, the upper and lower walls of the tubular portion are not limited to both being components of the vent pipe 35. For example, one of the two walls may be a component of the vent pipe 35, and the other may be a component of the base unit 30.

Furthermore, the outer surface of the cable CBL may be covered with a fluororesin. This improves the sliding of the cable CBL and suppresses undesired bending and buckling.
(1) A structure for wiring a cable to a rotating structure, the structure including: a non-rotating structure; a rotating structure which is arranged to be rotatable with respect to the non-rotating structure; and a cable which has one end connected to the non-rotating structure and the other end connected to the rotating structure, in which the non-rotating structure has a first cavity portion allowing deformation of a routing shape of the cable during rotation of the rotating structure, and the rotating structure has a main body having a side portion around which the cable is wound, a cover arranged separate from the side portion of the main body, and a second cavity portion formed between the main body and the cover and allowing deformation of the routing shape of the cable during the rotation of the rotating structure.
(2) The structure for wiring a cable to a rotating structure according to (1), in which the main body includes a cylindrical wall which is the side portion, and a connection terminal which is arranged at a position different from the second cavity portion in a direction orthogonal to a circumferential direction of the cylindrical wall and connects the cable in a direction different from the circumferential direction, and the rotating structure includes a cable fixing portion which bends the cable in a direction from the circumferential direction toward the connection terminal to fix the cable.
(3) The structure for wiring a cable to a rotating structure according to (1) or (2), in which the non-rotating structure has a first guide wall and a second guide wall which are arranged at an interval around the rotating structure, and a guide opening through which the cable is inserted between the first guide wall and the second guide wall, the first guide wall and the second guide wall have an arc shape on a side facing the guide opening, a first common circumscribed line which is in contact with each of a first virtual circle, the first virtual circle overlapping with the arc shape of the first guide wall, and a second virtual circle, the second virtual circle overlapping with the side portion in the main body of the rotating structure, does not overlap with the second guide wall, and a second common circumscribed line which is in contact with each of a third virtual circle, the third virtual circle overlapping with the arc shape of the second guide wall, and the second virtual circle does not overlap with the first guide wall.
(4) The structure for wiring a cable to a rotating structure according to (3), in which the first cavity portion and the second cavity portion are formed in regions of vertical angles with respect to an intersection point of the first common circumscribed line and the second common circumscribed line.
(5) The structure for wiring a cable to a rotating structure according to any one of (1) to (4), the structure including: a tubular portion which is positioned outside the cylindrical wall of the main body in plan view of the rotating structure and the non-rotating structure and which is formed by the rotating structure and the non-rotating structure, in which a height of the tubular portion is three times or less a width of the cable.
(6) The structure for wiring a cable to a rotating structure according to any one of (1) to (5), the structure including: a tubular portion which is positioned outside the cylindrical wall of the main body in plan view of the rotating structure and the non-rotating structure and which is formed by the rotating structure and the non-rotating structure, in which a width of the tubular portion is three times or less a width of the cable.
(7) The structure for wiring a cable to a rotating structure according to any one of (1) to (5), in which an outer surface of the cable is covered with a fluororesin.
(8) A CPAP device including: the structure for wiring a cable to a rotating structure according to any one of (1) to (7), in which the non-rotating structure is a main body which generates an air flow, and the rotating structure is a vent pipe.
10 CPAP device
20 main unit
30 base unit
31 box body
32 lid
35 vent pipe
36, 39 intake port
38 exhaust port
311 upper wall
312 lower wall
313, 314, 315, 316, 317 side wall
321 upper wall
323, 325, 326 side wall
350 vent hole
351 main body
352 cover
359 cavity portion
3210 opening
3211 main body cover
3290 peripheral wall
3291, 3292 guide wall
3297 protruding wall
3298 cable connection portion
3299 cavity portion
3511 substrate
3512 base
3513 main wall
3514 external connection wall
3516 external terminal housing
3517 internal connection terminal
3518 cable fixing portion
3519 vent pipe lid
3521 substrate
3522 base
3523 cylindrical body
3524 internal space
3528, 3529 protruding portion
B321 main body
C3512 concave portion
CBL cable
G329, G352 guide opening
Ls1 first common circumscribed line
Ls2 second common circumscribed line
Ps intersection point
S31A, S31B, S32 space

1. A structure for wiring a cable to a rotating structure, the structure comprising:
a non-rotating structure;
a rotating structure arranged to be rotatable with respect to the non-rotating structure; and
a cable having one end connected to the non-rotating structure and another end connected to the rotating structure, wherein
the non-rotating structure has a first cavity portion allowing deformation of a routing shape of the cable during rotation of the rotating structure, and
the rotating structure has
a main body having a side portion around which the cable is wound,
a cover arranged separate from the side portion of the main body, and
a second cavity portion arranged between the main body and the cover and allowing deformation of the routing shape of the cable during the rotation of the rotating structure.

2. The structure for wiring a cable to a rotating structure according to claim 1, wherein
the main body includes
a cylindrical wall being the side portion, and
a connection terminal arranged at a position different from the second cavity portion in a direction orthogonal to a circumferential direction of the cylindrical wall and connecting the cable in a direction different from the circumferential direction, and
the rotating structure includes a cable fixing portion configured to bend the cable in a direction from the circumferential direction toward the connection terminal to fix the cable.

3. The structure for wiring a cable to a rotating structure according to claim 1, wherein
the non-rotating structure has
a first guide wall and a second guide wall, the first guide wall and the second guide wall being arranged at an interval around the rotating structure, and
a guide opening through which the cable is inserted between the first guide wall and the second guide wall,
each of the first guide wall and the second guide wall has an arc shape on a side facing the guide opening,
a first common circumscribed line being in contact with each of a first virtual circle and a second virtual circle, the first virtual circle overlapping with the arc shape of the first guide wall, the second virtual circle overlapping with the side portion in the main body of the rotating structure, and the first common circumscribed line not overlapping with the second guide wall, and
a second common circumscribed line being in contact with each of a third virtual circle and the second virtual circle, the third virtual circle overlapping with the arc shape of the second guide wall, and the second common circumscribed line not overlapping with the first guide wall.

4. The structure for wiring a cable to a rotating structure according to claim 3, wherein
the first cavity portion and the second cavity portion are arranged in regions of vertical angles with respect to an intersection point of the first common circumscribed line and the second common circumscribed line.

5. The structure for wiring a cable to a rotating structure according to claim 1, the structure comprising:
a tubular portion positioned outside the cylindrical wall of the main body in plan view of the rotating structure and the non-rotating structure, and provided by the rotating structure and the non-rotating structure, wherein
a height of the tubular portion is three times or less a width of the cable.

6. The structure for wiring a cable to a rotating structure according to claim 1, the structure comprising:
a tubular portion positioned outside the cylindrical wall of the main body in plan view of the rotating structure and the non-rotating structure, and provided by the rotating structure and the non-rotating structure, wherein
a width of the tubular portion is three times or less a width of the cable.

7. The structure for wiring a cable to a rotating structure according to claim 1, wherein
an outer surface of the cable is covered with a fluororesin.

8. A CPAP device comprising:
the structure for wiring a cable to a rotating structure according to claim 1, wherein
the non-rotating structure is a main unit configured to generate an air flow, and
the rotating structure is a vent pipe.

9. The structure for wiring a cable to a rotating structure according to claim 2, wherein
the non-rotating structure has
a first guide wall and a second guide wall, the first guide wall and the second guide wall being arranged at an interval around the rotating structure, and
a guide opening through which the cable is inserted between the first guide wall and the second guide wall,
each of the first guide wall and the second guide wall has an arc shape on a side facing the guide opening,
a first common circumscribed line being in contact with each of a first virtual circle and a second virtual circle, the first virtual circle overlapping with the arc shape of the first guide wall, the second virtual circle overlapping with the side portion in the main body of the rotating structure, and the first common circumscribed line not overlapping with the second guide wall, and
a second common circumscribed line being in contact with each of a third virtual circle and the second virtual circle, the third virtual circle overlapping with the arc shape of the second guide wall, and the second common circumscribed line not overlapping with the first guide wall.

10. The structure for wiring a cable to a rotating structure according to claim 2, the structure comprising:
a tubular portion positioned outside the cylindrical wall of the main body in plan view of the rotating structure and the non-rotating structure, and provided by the rotating structure and the non-rotating structure, wherein
a height of the tubular portion is three times or less a width of the cable.

11. The structure for wiring a cable to a rotating structure according to claim 3, the structure comprising:
a tubular portion positioned outside the cylindrical wall of the main body in plan view of the rotating structure and the non-rotating structure, and provided by the rotating structure and the non-rotating structure, wherein
a height of the tubular portion is three times or less a width of the cable.

12. The structure for wiring a cable to a rotating structure according to claim 4, the structure comprising:
a tubular portion positioned outside the cylindrical wall of the main body in plan view of the rotating structure and the non-rotating structure, and provided by the rotating structure and the non-rotating structure, wherein
a height of the tubular portion is three times or less a width of the cable.

13. The structure for wiring a cable to a rotating structure according to claim 2, the structure comprising:
a tubular portion positioned outside the cylindrical wall of the main body in plan view of the rotating structure and the non-rotating structure, and provided by the rotating structure and the non-rotating structure, wherein
a width of the tubular portion is three times or less a width of the cable.

14. The structure for wiring a cable to a rotating structure according to claim 3, the structure comprising:
a tubular portion positioned outside the cylindrical wall of the main body in plan view of the rotating structure and the non-rotating structure, and provided by the rotating structure and the non-rotating structure, wherein
a width of the tubular portion is three times or less a width of the cable.

15. The structure for wiring a cable to a rotating structure according to claim 4, the structure comprising:
a tubular portion positioned outside the cylindrical wall of the main body in plan view of the rotating structure and the non-rotating structure, and provided by the rotating structure and the non-rotating structure, wherein
a width of the tubular portion is three times or less a width of the cable.

16. The structure for wiring a cable to a rotating structure according to claim 5, the structure comprising:
a tubular portion positioned outside the cylindrical wall of the main body in plan view of the rotating structure and the non-rotating structure, and provided by the rotating structure and the non-rotating structure, wherein
a width of the tubular portion is three times or less a width of the cable.

17. A CPAP device comprising:
the structure for wiring a cable to a rotating structure according to claim 2, wherein
the non-rotating structure is a main unit configured to generate an air flow, and
the rotating structure is a vent pipe.

18. A CPAP device comprising:
the structure for wiring a cable to a rotating structure according to claim 3, wherein
the non-rotating structure is a main unit configured to generate an air flow, and
the rotating structure is a vent pipe.

19. A CPAP device comprising:
the structure for wiring a cable to a rotating structure according to claim 4, wherein
the non-rotating structure is a main unit configured to generate an air flow, and
the rotating structure is a vent pipe.

20. A CPAP device comprising:
the structure for wiring a cable to a rotating structure according to claim 5, wherein
the non-rotating structure is a main unit configured to generate an air flow, and
the rotating structure is a vent pipe.
